(19) United States
(12) Patent Application Publication
(10) Pub. No.: US 2005/0010193 A1
(43) Pub. Date: Jan. 13, 2005

(54) Novel methods for administration of drugs and devices useful thereof

(76) Inventors: Philippe E. Laurent, Oullins (FR); Paul G. Alchas, Franklin Lakes, NJ (US); Ronald J. Pettis, Cary, NC (US)

Correspondence Address: JONES DAY, 222 East 41st Street, New York, NY, 10017, US

(21) Appl. No.: 10/704,035

(22) Filed: Nov. 6, 2003

Related U.S. Application Data
(63) Continuation-in-part of application No. 10/429,973, filed on May 6, 2003.
(60) Provisional application No. 60/377,649, filed on May 6, 2002.
(60) Provisional application No. 60/389,881, filed on Jun. 20, 2002.

(51) Int. Cl. A61M 31/00

(52) U.S. Cl. 604/506; 604/272

(57) ABSTRACT

The present invention relates to methods for administration of a substance into the junctional layer of a subject's skin, i.e., a transitory tissue between the reticular dermis and the hypodermis of the subcutaneous layer of the skin. The present invention provides an improved method of parenteral drug delivery in that it provides among other benefits, minimized unwanted immune response and inadvertent immunotoxic effects provoked by the substance administered. In addition, an improved pharmacokinetic profile can be obtained by employing the methods of the present invention. Devices that can be used in accordance with the methods of the invention are also disclosed.

This application is a continuation-in-part of U.S. application Ser. No. 10/429,973 filed May 6, 2003, which claims priority to U.S. Provisional Application Nos. 60/377,649, filed May 6, 2002, and 60/389,881, filed Jun. 20, 2002, all of which are incorporated herein in their entirety by reference.

### 1. FIELD OF THE INVENTION

The present invention relates to methods for administration of a substance into the junctional layer of a subject's skin, i.e., a transitory tissue between the reticular dermis and the hypodermis of the subcutaneous layer of the skin. The present invention provides an improved method of parenteral drug delivery in that it provides among other benefits, minimized unwanted immune response and inadvertent immunotoxic effects provoked by the substance administered. In addition, an improved pharmacokinetic profile can be obtained by employing the methods of the present invention. Devices that can be used in accordance with the methods of the invention are also disclosed.

### 2. BACKGROUND OF THE INVENTION

The importance of efficiently and safely administering pharmaceutical substances such as diagnostic agents and drugs has long been recognized. The use of conventional needles has long provided one approach for delivering pharmaceutical substances to humans and animals by administration through the skin. Considerable effort has been made to achieve reproducible and efficacious delivery through the skin while improving the ease of injection and reducing patient apprehension and/or pain associated with conventional needles. Furthermore, certain delivery systems eliminate needles entirely, and rely upon chemical mediators or external driving forces such as ionotophoretic currents or electroporation or thermal poration or sonophoresis to breach the stratum corneum, the outermost layer of the skin, and deliver substances through the surface of the skin. However, such delivery systems do not reproducibly breach the skin barriers or deliver the pharmaceutical substance to a given depth below the surface of the skin and consequently, clinical results can be variable. Thus, mechanical breach of the stratum corneum such as with needles, is believed to provide the most reproducible method of administration of substances through the surface of the skin, and to provide control and reliability in placement of administered substances.

Approaches for delivering substances beneath the surface of the skin have almost exclusively involved transdermal administration, i.e., delivery of substances through the skin to a site beneath the skin. Transdermal delivery includes subcutaneous, intramuscular or intravenous routes of administration of which, intramuscular and subcutaneous injections have been the most commonly used.

Anatomically, the outer surface of the body is made up of two major tissue layers, an outer epidermis and an underlying dermis, which together constitute the skin (for review, see Physiology, Biochemistry, and Molecular Biology of the Skin, Second Edition, L. A. Goldsmith, Ed., Oxford University Press, New York, 1991). The epidermis is subdivided into five layers or strata of a total thickness of between 75 and 150 μm. Beneath the epidermis lies the dermis, which contains two layers, an outermost portion referred to as the papillary dermis and a deeper layer referred to as the reticular dermis. The papillary dermis contains vast microcirculatory blood and lymphatic plexuses. In contrast, the reticular dermis is relatively acellular and avascular and made up of dense collagenous and elastic connective tissue. Beneath the epidermis and dermis is the subcutaneous tissue, also referred to as the hypodermis, which is composed of connective tissue and fatty tissue. Muscle tissue lies beneath the subcutaneous tissue. The reticular dermis and subcutaneous tissue are separated by an interface, called a junctional layer.

The junctional layer is bordered by several collagen bundles, which form the reticular dermis just above the junctional layer. These bundles are arranged in a parallel fashion to the skin surface, anchoring the elastic fibers of the papillary dermis. In the junctional layer, fibrous connective tissue that consists of smooth, flexible and dispersed collagen fibers are perpendicularly oriented from the reticular dermis. This connective tissue is associated with glycoaminoglycans and proteoglycans, and supports fibroblasts, a few adipose cells and infiltrating cells from blood vessels. Also characteristic of the junctional layer is the presence of a dense network of blood vessels resolving in capillary loops of the dermis. Importantly, the deep venous plexus collecting the post-capillary veins are located in the junctional layer. Under the skin junctional layer is the adipose tissue that forms the subcutaneous layer. The thickness of the junctional layer is estimated between 1.9 to 3 mm. This data is based on clinical investigation in healthy volunteers using X-ray CT scanning and high frequency (20 MHz) ultrasounds imaging techniques.

As noted above, both the subcutaneous tissue and muscle tissue have been commonly used as sites for administration of pharmaceutical substances. One of the most serious problems encountered in administrating a pharmaceutical substance into these compartments, however, is the potential risk of evoking an unwanted immune response in the subject. In some cases, such an unwanted immune response can lead to the death of the subject. Therefore, while there is an on-going need for methods of administration of a substance that results in an improved pharmacokinetics, a specific need for methods of administration of a substance that can reduce the potential harmful effects caused by the administration, such as an unwanted immune response, also exists.

### 3. SUMMARY OF THE INVENTION

The present invention provides a new parenteral administration method by selectively and specifically targeting the junctional layer of a subject's skin, thereby resulting in enhanced therapeutic efficacy of the delivered substance. Substances delivered in accordance with the methods of the invention have an improved clinical utility and therapeutic efficacy relative to other drug delivery methods including intradermal and subcutaneous delivery. The present invention provides benefits and improvements over conventional drug delivery methods including but not limited to improved pharmacokinetics, reduction of undesired and harmful side-effects, reduction or elimination of pain perception by the subject, and absence of limitation on the volume of injection.

The present invention is based, in part, on the inventors' unexpected discovery that delivering a substance to the junctional layer of a subject's skin provides an improved delivery method. As used herein, junctional layer refers to the transitory tissue space between the deepest layer of the dermis, i.e., the reticular dermis, and the hypodermis of the subcutaneous layer of the skin.

As used herein, administration into the junctional layer is intended to encompass administration of a substance into the junctional layer in such a manner that the substance is deposited in the junctional layer such that it readily reaches the dense network of venous plexus and postcapillary veins of the junctional layer, and is rapidly absorbed and systemically distributed. In accordance with the methods of the invention, deposition of a substance into the junctional layer occurs predominately at a depth of at least about 1.5 mm, preferably, at least about 2 mm, up to a depth of no more than about 3 mm, preferably, no more than about 2.5 mm, which results in rapid absorption of the substance and reduced immune response. Therefore, the substances delivered in accordance with the methods of the invention may exert their beneficial effects more rapidly than other routes of administration, including ID and IM.

Preferably, substances delivered in accordance with the methods of the invention penetrate the junctional layer of the subject's skin without passing through it. Placement of the substance into the subcutaneous layer (e.g., at a depth greater than 2.5 mm) may not only result in slower absorption of the substance but may also be associated with unwanted immune response, and is thus undesirable in accordance with the methods of the instant invention.

The present invention provides for targeting and deposition of a substance into the junctional layer of the skin. Delivering a substance into a subject's junctional layer in accordance with the methods of the invention results in improved pharmacokinetics, e.g., an improved pharmacokinetic profile. Although not intending to be bound by a particular theory, it is believed that due to the dense network of venous plexus and post-capillary veins that infiltrate the junctional layer, administering the substance directly into the junctional layer would result in a more efficient uptake of the substance than subcutaneous or intramuscular administration, which, in turn, would result in an improved pharmacokinetics. Furthermore, by specifically and selectively targeting the junctional layer for the delivery, the pharmacokinetics exhibited by the substance is consistently reproducible, resulting in a reduced inter-individual variability of PK parameters relative to other conventional delivery methods.

The methods of the present invention not only provide improved pharmacokinetics over conventional drug delivery methods, but also provide additional benefits including a reduction in harmful side effects caused by the administration of a substance, such as an unwanted immune response and inadvertent immuno-toxic effect against the active ingredients of the substance. As used herein, the term “unwanted immune response” means the natural immune response of the subject receiving a substance of this invention, where the substance is not intended to provoke such response when administered. Examples of unwanted immune responses that may be prevented using the methods of the invention include, but are not limited to, IgE-mediated hypersensitivity, with the risk of local and/or systemic anaphylactic reaction as described, for instance, after parenteral injection of insulin or heparin and many other drugs based on having a protein or a polysaccharide as the active ingredient; antibody-mediated cytotoxic hypersensitivity as well as immune complex mediated hypersensitivity that cause systemic adverse events, such as kidney and/or liver and/or microvascular alteration due to the deposition of circulating immune complexes; cell-mediated hypersensitivity with the risk of inducing delayed type reaction at the injection site and immune neutralization of the active ingredient, or any systemic adverse event, such as thrombocytopenia induced by heparin treatment.

The methods of the invention are thus particularly useful for the delivery of therapeutic substances to which the induction of an immune response would not be beneficial to the therapeutic effect of the substance to be delivered. Examples of such substances include low molecular weight heparins, pentasaccharides, interferon alpha and beta, erythropoeitines, antibodies, polypeptidic hormones, growth hormone, and interleukins. The reduced risk of immuno-toxic effect in accordance with the delivery methods of the invention is due, in part, to the low preponderance of immunocompetent cells in the junctional layer. Therefore, the methods of the invention are preferred over intradermal delivery of such substances, where the risk of unwanted immune response is higher since the intradermal space is characterized by a high concentration of immunocompetent cells, e.g., dendritic cells, monocytes, lymphocytes, macrophages, etc. In a specific embodiment, the substance to be administered in accordance with the methods of the invention is not a vaccine, for which minimizing immune response may be unfavorable.

Delivering a substance in accordance with the methods of the invention reduces or eliminates pain perceived by the subject. Therefore, the methods of the instant invention are preferred to other parenteral delivery methods, including intradermal delivery. Although not intending to be bound by a particular mechanism of action, the intradermal layer is a sensory organ characterized by nerve endings and nervous corpuscles. In contrast, the junctional layer has poor nerve endings and sensory corpuscles, and thus the pain perception induced by the administration of a substance into the junctional layer is lower than that perceived by delivering the substance to the intradermal layer.

Another benefit of delivering a substance in accordance with the methods of the invention is the absence of limitation on the injection volume of the substance compared to delivering the substance into other tissue compartments. Therefore, the methods of the instant invention are particularly advantageous over intradermal delivery, whereby the volume of the injected substance is limited to about 50 to 250 μL due in part to the high network of collagen bundles and elastin fibers of the dermal layer and the dermal tissue deformability. Although not intending to be bound by a particular mechanism due to the flexibility and high deformability of the junctional connective tissue there is no limitation on the volume of injection when delivering a substance to the junctional layer, particularly by bolus injection. The volume of the substance that may be used in the methods of the invention may be the same injection volume as those for subcutaneous administration. Therefore, using the methods of the present invention, injection volume of the substance may be about 0.5 mL or more, more specifically about 1.0 mL or more.

The present invention encompasses any device for accurately and selectively targeting the junctional layer of a subject's skin. The nature of the device used is not critical as long as it penetrates the skin of the subject to the targeted depth within the junctional region without passing through it. Preferably, the device penetrates the skin at a depth of at least about 2 mm, up to a depth of no more than about 3 mm, most preferably, no more than about 2.5 mm.

In some embodiments, the present invention encompasses delivering a substance into the junctional layer of a subject's skin using a device that comprises at least one needle, preferably a microneedle. Preferably, the needle has a length sufficient to penetrate the junctional layer and an outlet at a depth within the junctional layer so that the substance is delivered and distributed in the junctional layer. In some embodiments, the length of the needle is about 2 mm to about 5 mm, preferably about 2 mm to about 3 mm. In other embodiments, the outlet of the needle is placed at a depth of about 2 mm to about 3 mm, preferably about 2 mm to about 2.5 mm, when the needle is inserted.

The invention encompasses pharmaceutical formulations comprising one or more substances for junctional delivery. In some embodiments, the formulations containing a substance of the invention comprises a therapeutically or prophylactically effective amount of the substance. In other embodiments the formulations of the invention comprise one or more other additives. The formulations to be administered according to the methods of the present invention may be in any form suitable for junctional delivery.

Using the methods of the present invention, a substance may be administered as a bolus, or by infusion. As used herein, the term “bolus” is intended to mean an amount that is delivered within a time period of less than ten (10) minutes. “Infusion” is intended to mean the delivery of a substance over a time period greater than ten (10) minutes. It is understood that bolus administration or delivery can be carried out with rate controlling means, for example a pump, or have no specific rate controlling means, for example user self-injection.

### 4. BRIEF DESCRIPTION OF FIGURES

FIG. 1 ANATOMY OF SKIN: Various layers of the skin with their respective boundaries are indicated schematically

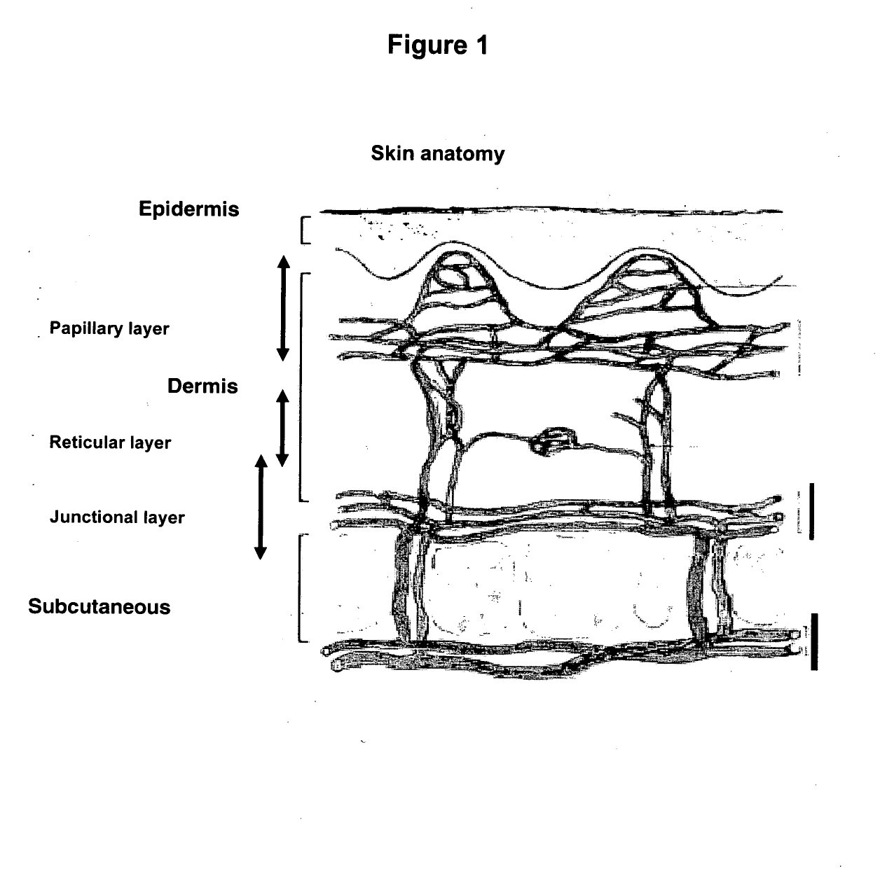

FIG. 2 INJECTION DEVICES: The needle lengths of a delivery device is dependent on the compartment of skin being targeted. Optimal needle lengths for intradermal, junctional and shallow subcutaneous injections are 1.5 mm, 2-3 mm, and 4-5 mm, respectively.

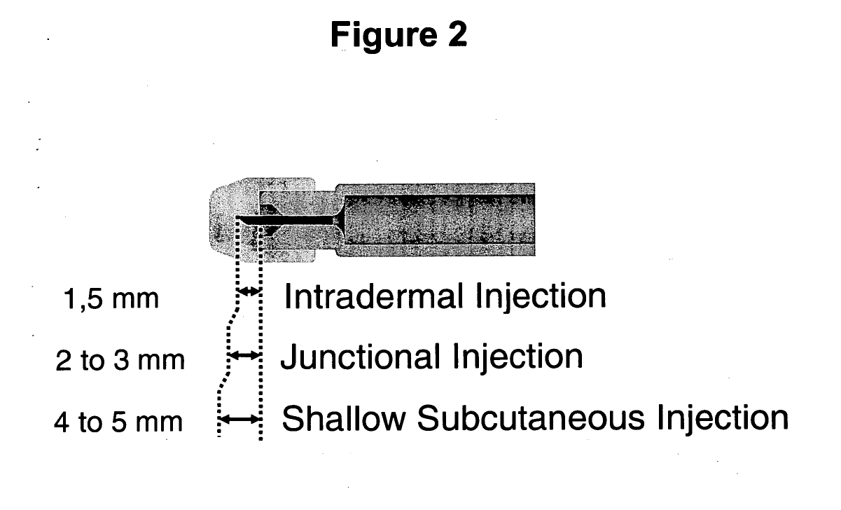

FIG. 3 GEOMETRIC MEAN TITERS OF ANTIBODY: Antibody titers at D0, D7, D14 resulting from a single dose of rabies vaccine are compared between different routes of injection. Rabies vaccine was administered by IM, ID or junctional route.

FIG. 4 GEOMETRIC MEAN TITERS OF ANTIBODY: Antibody titers after two and three subsequent injections were monitored over time. Rabies vaccine was administered by IM, ID or junctional route.

### 5. DETAILED DESCRIPTION OF THE INVENTION

The present invention is directed, in part, to a method for administration of a substance to a subject's skin, comprising delivering the substance, preferably, selectively and specifically, into the junctional layer of the subject's skin. In one embodiment, the subject is a human or an animal, preferably a human. As used herein, junctional layer refers to the transitory tissue space between the deepest layer of the dermis, i.e., the reticular dermis, and the hypodermis of the subcutaneous layer. The junctional layer is bordered by several collagen bundles, which form the reticular dermis just above the junctional layer. In the junctional layer, fibrous connective tissue that consists of smooth, flexible and dispersed collagen fibers are perpendicularly oriented from the reticular dermis. Also characteristic of the junctional layer is the presence of a dense network of blood vessels resolving in capillary loops of the dermis. Importantly, the deep venous plexus collecting the post-capillary veins are located in the junctional layer.

The present invention provides a new parenteral administration method by selectively and specifically targeting the junctional layer of a subject's skin, thereby resulting in enhanced therapeutic efficacy of the delivered substance. In some embodiments, the junctional layer is directly targeted. As used herein, administration into the junctional layer is intended to encompass administration of a substance into the junctional layer in such a manner that the substance is deposited in the junctional layer such that it readily reaches the dense network of venous plexus and postcapillary veins of the junctional layer, and is rapidly absorbed and systemically distributed. In accordance with the methods of the invention, deposition of a substance into the junctional layer occurs predominately at a depth of at least about 1.5 mm, preferably at least about 2 mm, up to a depth of no more than about 3 mm, preferably no more than about 2.5 mm, which results in rapid absorption of the substance and reduced immune response. Therefore, the substances delivered in accordance with the methods of the invention may exert their beneficial effects more rapidly than other routes of administration, including SC and IM.

The methods of the invention are beneficial over traditional drug delivery methods, by for example, improving the pharmacokinetics of the administered substance, reduction of undesired and harmful side effects, reduction or elimination of pain perceived, and the absence of volume limitation on the volume of the injection.

Substances administered using the method of the present invention yield pharmacokinetics superior to, and more clinically desirable than that obtained for the same substance administered by conventional methods of delivery. Although not intending to be bound by a particular theory, it is believed that due to the dense network of venous plexus and post-capillary veins that infiltrate the junctional layer, administering the substance directly into the junctional layer would result in a more efficient uptake of the substance than subcutaneous or intramuscular administration, which, in turn, would result in an improved pharmacokinetics. Furthermore, by specifically and selectively targeting the junctional layer, the pharmacokinetics exhibited by the substance is consistently reproducible, resulting in reduced inter-individual variability of PK parameters relative to other conventional delivery methods.

According to the present invention, improved pharmacokinetics means increased bioavailability, decreased lag time (T_{lag}), decreased Tₘₐₓ, more rapid absorption rates, more rapid onset and/or increased Cₘₐₓ for a given amount of substance administered, compared to conventional administration methods.

By bioavailability, it is meant the total amount of a given dosage of the administered substance that reaches the blood compartment. This is generally measured as the area under the curve in a plot of concentration vs. time. By “lag time,” it is meant the delay between the administration of the substance and time to measurable or detectable blood or plasma levels. Tₘₐₓ is a value representing the time to achieve maximal blood concentration of the substance, and Cₘₐₓ is the maximum blood concentration reached with a given dose and administration method. The time for onset is a function of T_{lag}, Tₘₐₓ and Cₘₐₓ, as all of these parameters influence the time necessary to achieve a blood (or target tissue) concentration necessary to realize a biological effect. Tₘₐₓ and Cₘₐₓ can be determined by visual inspection of graphical results and can often provide sufficient information to compare two methods of administration of a substance. However, numerical values can be determined more precisely by kinetic analysis using mathematical models and/or other means known to those of skill in the art.

The measurement of pharmacokinetic parameters and determination of minimally effective concentrations are routinely performed in the art. Values obtained are deemed to be enhanced by comparison with a standard route of administration such as, for example, subcutaneous, intradermal or intramuscular administration. In such comparisons, it is preferable, although not necessarily essential, that administration into the junctional layer and administration into the reference site such as subcutaneous administration involve the same dose levels, i.e., the same amount and concentration of drug as well as the same carrier vehicle and the same rate of administration in terms of amount and volume per unit time. Thus, for example, administration of a given pharmaceutical substance into the junctional layer at a concentration such as 100 μg/mL and rate of 100 μL per minute over a period of 5 minutes would, preferably, be compared to administration of the same pharmaceutical substance into the subcutaneous space at the same concentration of 100 μg/mL and rate of 100 μL per minute over a period of 5 minutes.

The methods of the present invention not only provide improved pharmacokinetics of the delivered substance compared to traditional drug delivery methods but also provide additional benefits, including, a reduction of the harmful side effects caused by the administration of a substance, such as an unwanted immune response and inadvertent immunotoxic effect against the active ingredients of the substance. As used herein, the term “unwanted immune response” means the natural immune response of the subject receiving a substance of this invention, where the substance is not intended to provoke such response when administered. Examples of unwanted immune responses that may be prevented using the methods of the invention include, but are not limited to, IgE-mediated hypersensitivity, antibody-mediated cytotoxic hypersensitivity, immune complex mediated hypersensitivity, and cell mediated hypersensitivity, immune neutralization of the active ingredient by antibodies and finally cross-reactivity of the antibodies formed against the active ingredient against natural compounds sharing the same antigenic motifs as the substance administered.

The methods of the invention are thus particularly useful for therapeutic substances to which the induction of an immune response would not be beneficial to the therapeutic effect of the substance to be delivered. Examples of therapeutic substances for use in the methods of the invention, include low molecular weight heparins, pentasaccharides, interferon alpha and beta, erythropoeitines, antibodies, polypeptidic hormones, growth hormone, and interleukins. Therapeutic substances used in the methods of the invention include recombinant proteins. Additional non-limiting examples of substances that may be used in the methods of the invention are disclosed herein in Section 5.1. The reduced risk of immuno-toxic effect in accordance with the delivery methods of the invention is due in part to the low preponderance of immunocompetent cells in the junctional layer. Therefore the methods of the invention are preferred over intradermal delivery of such substances, where the risk of unwanted immune response is higher since the intradermal space is characterized by a high concentration of immunocompetent cells, e.g., dendritic cells, monocytes, lymphocytes, macrophages, etc. In a specific embodiment, the substance to be administered in accordance with the methods of the invention is not a vaccine, for which minimizing immune response may be unfavorable.

Furthermore, selectively targeting the junctional layer for the delivery of substances can result in other clinical benefits, such as reduced pain perception. Although not intending to be bound by a theory since the junctional layer has poor nerve endings and sensory corpuscles, the pain perception induced by the administration of a substance into the junctional layer may be lower than that perceived by administration to other tissue compartments. Delivering a substance in accordance with the methods of the invention reduces or eliminates pain perceived by the subject. Therefore, the methods of the instant invention are preferred to other drug delivery methods, including intradermal delivery.

Another benefit of delivering a substance in accordance with the methods of the invention is the absence of limitation on the injection volume of the substance compared to delivering the substance into other tissue compartments. Therefore, the methods of the instant invention are particularly advantageous to intradermal delivery, whereby the volume of the injected substance is limited to about 50 to 250 μL due, in part, to the high network of collagen bundles and elastin fibers of the dermal layer and the dermal tissue deformability. Although not intending to be bound by a particular mechanism due to the flexibility and high deformability of the junctional connective tissue there is no limitation on the volume of injection when targeting the junctional layer, particularly bolus injection. The volume of the substance that may be used in the methods of the invention may be the same injection volume as those for subcutaneous administration. Therefore, using the methods of the present invention, injection volume of the substance may be about 0.5 mL or more, more specifically about 1.0 mL or more.

The methods of the instant invention thus provide methods for treatment, prevention, or amelioration of one or more symptoms associated with a disease, disorder or infection by delivering one or more substances of the invention to the junctional layer of a subject's skin. Substances delivered in accordance with the methods of the invention have enhanced clinical utility and therapeutic efficacy relative to other delivery methods.

5.1 Substances for Administration

The present invention encompasses the administration of a wide variety of substances by selectively targeting them into a subject's junctional layer. Examples of substances that may be administered using the method of the present invention include, but are not limited to, pharmaceutically or biologically active substances including diagnostic agents, drugs, and other substances which provide therapeutic or health benefits, such as, but not limited to, neutriceuticals. The invention encompasses the administration of any protein, particularly a therapeutic protein, and all salts, polymorphs, analogs, derivatives, fragments, mimetics and peptides thereof, which can be obtained using standard methods known to one skilled in the art.

Substances that are particularly suited for the methods of the invention are which can benefit from a reduced risk of unwanted immune response and immuno-toxic effects and those which can benefit from an improved pharmacokinetic profile, including but not limited to low molecular weight heparins, pentasaccharides, interferon alpha and beta, erythropoeitines, antibodies, polypeptidic hormones, growth hormone, and interleukins.

The methods of the instant invention are particularly useful for delivery of anti-thrombosis agents, such as low molecular weight heparins and synthetic pentasaccharides. These substances can immensely benefit from reduced unwanted immune response achieved by the methods of the present invention. Adverse immune reactions have been documented in the art for administration of low molecular weight heparins, such as Enoxaparin®. For example, there has been reports of hypersensitivity reactions upon SC administration of Enoxaparin® (See, e.g., Pharmacotherapy, 2002, 22 (11): 1511-5) and delayed type hypersensitivity (DTH) reaction in patients receiving LMW heparin therapy (See, e.g., Dermatol. Surg. 2001 27 (1): 47-52). Additionally, heparin induced thrombocytopenia (HIT) has been reported as a major side effect of heparin administration; HIT is a serious and potentially life threatening syndrome, which is a result of antibodies formed against Platelet factor 4/heparin complex. The occurrence of HIT has been estimated in 1-3% of all patients receiving heparin therapy. (See, Perfusion, 2003, 18 (1): 47-53; Eur. J. Pediatr. 158 (Suppl. 3): S130-133 (1999). Although synthetic pentasaccharides, such as Fondaparinux®, have been reported to be associated with a lower immuno-toxic risk than low molecular weight heparins, a significant potential risk still exists. (Clin. Ther. 24 (11): 1757-1769 (2002). Therefore, the methods of the invention provide alternative and more improved methods for antithrombotic therapy, since delivering the antithrombotic agents such as heparins, including low molecular weight heparins, reduces or eliminates, the undesired immune responses that are associated with current antithrombotic therapy, such as DTH and HIT.

The invention encompasses delivering any of the agents used for the treatment and/or prevention of thrombosis related disorders. Four main types of therapies are used to prevent or treat thrombosis: antiplatelet agents, anticoagulant agents (heparin), vitamin K antagonists (coumarin derivatives) and thrombolytic agents. Each type of agent interferes with clotting at a different site in the coagulation pathway (See, Goodman & Gilman, The Pharmacological Basis of Therapeutics, 9th ed., McGraw-Hill, NY (1996)). Dipyridamole is another agent sometimes used to prevent or treat thrombosis; it is a vasodilator that, in combination with warfarin (a coumarin derivative), inhibits embolization from prosthetic heart valves and, in combination with aspirin, reduces thrombosis in patients with thrombotic disorders. The invention also encompasses inhibitors of the cell surface glycoprotein GP IIb/IIIA, which belong to a new family of anti-thrombosis agents mostly indicated in coronary diseases as well as thrombin inhibitors which have been characterized to have anti-thrombosis effect.

The methods of the invention are useful for treating and/or preventing any thrombosis related disorder including, but not limited to, deep vein thrombosis, pulmonary embolism, thrombophlebitis, arterial occlusion from thrombosis or embolism, arterial reocclusion during or after angioplasty or thrombolysis, restenosis following arterial injury or invasive cardiological procedures, postoperative venous thrombosis or embolism, acute or chronic atherosclerosis, stroke, myocardial infarction, cancer and metastasis, and neurodegenerative diseases.

Diagnostic substances that may be used in accordance with the method of the present invention include, but are not limited to, insulin, ACTH (e.g., corticotropin injection), luteinizing hormone-releasing hormone (e.g., Gonadorelin Hydrochloride), growth hormone-releasing hormone (e.g., Sermorelin Acetate), cholecystokinin (Sincalide), parathyroid hormone and fragments thereof (e.g., Teriparatide Acetate), thyroid releasing hormone and analogs thereof (e.g., protirelin), secretin and the like.

Therapeutic substances that may be used with the present invention include, but are not limited to alpha-1 anti-trypsin; anti-angiogenesis agents; antisense agents; butorphanol; calcitonin and analogs; ceredase; Cox-II inhibitors; dermatological agents; dihydroergotamine; dopamine agonists and antagonists; enkephalins and other opioid peptides; epidermal growth factors; erythropoietin and analogs; follicle stimulating hormone; G-CSF; glucagon; GM-CSF; granisetron; growth hormone and analogs (including growth hormone-releasing hormone); growth hormone antagonists; heparins; hirudin and hirudin analogs such as hirulog; IgE suppressors; insulin; insulinotropin and analogs; insulin-like growth factors; interferons; interleukins; luteinizing hormone; luteinizing hormone-release hormone and analogs; low molecular weight heparins and other natural modified or synthetic glycoaminoglycans; M-CSF; metoclopramide; midalzolam; monoclonal antibodies, pegylated antibodies, pegylated proteins or any proteins modified with hydrophilic or hydrophobic polymers or additional functional groups, fusion proteins, single chain antibody fragments or the same with any combination of attached proteins, macromolecules or additional functional groups thereof, narcotic analgesics; nicotine; non-steroid anti-inflammatory agents; oligosaccharides; ondansetron; parathyroid hormone and analogs; parathyroid hormone antagonists, prostaglandin antagonists; prostaglandins; recombinant soluble receptors; scopolamine; serotonin agonists and antagonists; sildenafil; terbutaline; salbutanol; modafinil; thrombolytics; tissue plasminogen activators; TNF and its antagonists; and vaccines.

In some embodiments, substances that may be administered using the methods of the present invention are those whose desired profile dictates a rapid onset of action followed by a longer circulating levels of the drug. An example of such a substance is insulin, for which a rapid and high peak onset levels is desired to cover the high glucose levels obtained from digestion and absorption of sugars or other non-complex carbohydrates, and meanwhile it is desired to rapidly bring blood glucose back to normal level. The invention encompasses use of insulin, glucagon-like peptides, all salts, polymorphs, analogs, derivatives, fragments, mimetics and peptides thereof. Another example of substances useful in the methods of the invention are pain relief agents (e.g., Cox inhibitors, morphine, opioids and other narcotic analgesics and triptans); erectile dysfunction agents, e.g., sildenafil; anti-clotting factors (e.g., heparin, low molecular weigh heparin, GPIIb/IIa antagonist, fondaparine); anti acute-anxiety disorders such as panic attack (e.g., midazolan, diazepam, tricyclic); acute day-time sleepiness attack (e.g., modafinil; seizure (e.g., diazepam). Moreover, a faster absorption may be favorable for substances that generally have a slow absorption when administered by conventional delivery methods, e.g., SC. For traditional SC administration high molecular weight drugs are slowly absorbed since absorption is an inverse function of the molecular weight. Examples of such substances include, but are not limited to, high molecular weight or hydrophobic drug compounds.

5.2 Formulations for Junctional Delivery

The invention encompasses formulations comprising one or more substances for junctional delivery. In some embodiments, the formulations containing a substance of the invention comprise a therapeutically or prophylactically effective amount of the substance. In other embodiments, the formulations of the invention comprise one or more other additives.

As used herein, and unless otherwise specified, a “therapeutically effective amount” refers to an amount of a substance of the present invention or other active ingredient sufficient to provide a therapeutic benefit in the treatment or management of the disease or to delay or minimize symptoms associated with the disease. Further, a therapeutically effective amount with respect to a substance of the invention means that amount alone, or in combination with other therapies, provides a therapeutic benefit in the treatment or management of the disease. Used in connection with an amount of a substance of the present invention, the term can encompass an amount that improves overall therapy, reduces or avoids symptoms or causes of disease, or enhances the therapeutic efficacy of or synergies with another therapeutic agent.

As used herein, and unless otherwise specified, a “prophylactically effective amount” refers to an amount of a substance of the invention or other active ingredient sufficient to result in the prevention, recurrence or spread of the disease. A prophylactically effective amount may refer to the amount sufficient to prevent initial disease, the recurrence or spread of the disease or the occurrence of the disease in a patient, including but not limited to those predisposed to the disease. A prophylactically effective amount may also refer to the amount that provides a prophylactic benefit in the prevention of the disease. Further, a prophylactically effective amount with respect to a substance of the invention means that amount alone, or in combination with other agents, provides a prophylactic benefit in the prevention of the disease. Used in connection with an amount of a substance of the invention, the term can encompass an amount that improves overall prophylaxis or enhances the prophylactic efficacy of or synergies with another prophylactic agent.

Additives that may be used in the formulations containing a substance of the invention include for example, wetting agents, emulsifying agents, or pH buffering agents. The formulations containing a substance of the invention may contain one or more other excipients such as saccharides and polyols. Preferably, the pharmaceutically acceptable carrier does not itself induce a physiological response, e.g., an immune response. Most preferably, the pharmaceutically acceptable carrier does not result in any adverse or undesired side effects and/or does not result in undue toxicity. Pharmaceutically acceptable carriers for use in the formulations of the invention include, but are not limited to, saline, buffered saline, dextrose, water, glycerol, sterile isotonic aqueous buffer, and combinations thereof. Additional examples of pharmaceutically acceptable carriers, diluents, and excipients are provided in Remington's Pharmaceutical Sciences (Mack Pub. Co., N.J., current edition; all of which is incorporated herein by reference in its entirety).

The formulations of the invention can be a solid, such as a lyophilized powder suitable for reconstitution, a liquid solution, a suspension, a tablet, a pill, a capsule, a sustained release formulation, or a powder.

The formulations containing a substance of the present invention can be prepared using any accepted methods of preparation known in the art. The specific method of preparation depends on the specific substance to be administered, and such variations are within the ordinary skill in the art.

The invention encompasses methods of administering solution and particulate forms of a substance of the invention and mixture thereof, including fast-acting, intermediate-acting, and long-acting formulations that may be obtained from any substances. The formulations used in the methods and formulations of the invention may be a mixture of one or more formulations that contain a substance of the present invention.

The substances in the formulations may be in different physical association states, for example, monomeric or dimeric states. The chemical state of the substance may be modified by standard recombinant DNA technology to produce the substance of different chemical formulas in different association states. Alternatively solution parameters, such as pH, may be altered to result in formulations of the substance in different association states. Other chemical modifications of the substances of the invention are also encompassed by the instant invention.

Using the methods of the invention lower doses of substances are required to achieve a similar therapeutic efficacy as conventional methods of administration. The substances delivered in accordance with the methods of the invention have improved therapeutic efficacy compared to the same substances delivered by conventional methods.

In the case that biological molecules are to be administered, such molecules may be from different animal species including, limited but not to, swine, bovine, ovine, equine, etc.

The invention encompasses formulations in which a substance of the present invention is in a particulate form, i.e., is not fully dissolved in solution. In some embodiments, at least 30%, at least 50%, at least 75% of the substance is in particulate form. Although not intending to be bound by a particular mode of action, formulations of the invention in which the substance is in particulate form have at least one agent which facilitates the precipitation of the substance. Precipitating agents that may be employed in the formulations of the invention may be proteinacious, e.g., protamine, a cationic polymer, or non-proteinacious, e.g., zinc or other metals or polymers.

The form of a substance to be delivered or administered include solutions thereof in pharmaceutically acceptable diluents or solvents, emulsions, suspensions, gels, particulates such as micro- and nanoparticles, either suspended or dispersed, as well as in-situ forming vehicles of the same. The formulations containing a substance of the invention may be in any form suitable for junctional delivery. In one embodiment, the junctional formulation of the invention is in the form of a flowable, injectible medium, i.e., a low viscosity formulation that may be injected in a syringe. The flowable injectible medium may be a liquid. Alternatively the flowable injectible medium is a liquid in which particulate material is suspended, such that the medium retains its fluidity to be injectible and syringable, e.g., can be administered in a syringe.

The formulations of the present invention can be prepared as unit dosage forms. A unit dosage per vial may contain 0.1 to 1 mL of the formulation. In some embodiments, a unit dosage form of the junctional formulations of the invention may contain about 50 μL to 100 μL, 50 μL to 200 μL, 50 μL to 500 μL or 50 μL to 1 mL of the formulation. If necessary, these preparations can be adjusted to a desired concentration by adding a sterile diluent to each vial.

The volumes of the formulations administered in accordance with the methods of the invention are not administered in volumes whereby the junctional layer might become overloaded leading to partitioning to one or more other compartments, such as the subcutaneous compartment. However, the volume of the formulation when using the junctional administration of the present invention is much less critical that when other conventional administration methods are used. Without being bound by a particular theory, it is believed that the junctional injection is much more receptive to a larger volume bolus due to the flexibility and high deformability of the junctional connective tissue. Therefore, using the methods of the present invention, injection volume of about 0.5 mL or greater, more specifically about 1.0 mL or greater, may be administered into the junctional layer.

5.3 Methods for Junctional Delivery

In some embodiments, the present invention encompasses methods for junctional delivery of substances described and exemplified herein to the junctional layer of a subject's skin, preferably by selectively and specifically targeting the junctional layer without passing through it. In a most preferred embodiment, the junctional layer is targeted directly. Once a formulation containing the substance to be delivered is prepared, the formulation is typically transferred to an injection device for junctional delivery, e.g., a syringe. The invention is based, in part, on the inventors' discovery that delivery of a substance to the junctional layer reduces or eliminates undesired immune responses and immunotoxic effects, including but not limited to, IgE-mediated hypersensitivity, antibody-mediated cytotoxic hypersensitivity, immune complex mediated hypersensitivity, and cell mediated hypersensitivity, immuno-neutralization of active ingredient and/or risk of immune cross-reactivity of antibody and/or cell-mediated immunity against natural substances. Delivery of the formulations of the invention in accordance with the methods of the invention provides an improved therapeutic and clinical efficacy of the substance. The formulations containing the substances of the present invention have improved pharmacokinetics such as an improved absorption uptake within the junctional layer.

As used herein, administration into the junctional layer is intended to encompass delivery of a substance into the junctional layer in such a manner that the substance readily reaches the dense network of venous plexus and postcapillary veins of the junctional layer, and is rapidly absorbed and becomes systemically bioavailable. It is believed that deposition of a substance predominately at a depth of at least about 1.5 mm, preferably, at least about 2 mm, up to a depth of no more than about 3 mm, preferably, no more than about 2.5 mm, will result in rapid absorption of the substance and reduced immune response. Preferably, substances delivered in accordance with the methods of the invention penetrate the junctional layer of the subject's skin without passing through it. Placement of the substance into the subcutaneous layer (e.g., at a depth greater than 2.5 mm) may not only result in slower absorption of the substance but may also be associated with unwanted immune response, and is thus undesirable in accordance with the methods of the instant invention.

The actual method by which the formulation is targeted to the junctional layer is not critical as long as it penetrates the skin of a subject to the desired targeted depth within the junctional layer without passing through it. In most cases, the device will penetrate the skin and to a depth of about 2-3 mm, preferably at a depth of 2.5 mm. In some embodiments the device will penetrate the skin to a depth no more than 2.5 mm. One of the advantages of delivering a substance to the junctional layer in accordance with the methods of the invention is that the thickness of the junctional layer does not depend on the site of the injection or on the particular subject. Any injection site for junctional administration may be used in the methods of the invention, including, but not limited to, the junctional layer of thigh, abdomen, pectoral or chest deltoid, forearm and back of the forearm.

The junctional methods of administration comprise microneedle-based injection and infusion systems or any other means to accurately target the junctional layer. The junctional methods of administration encompass not only microdevice-based injection means, but other delivery methods such as needle-less or needle-free ballistic injection of fluids or powders into the junctional layer, enhanced ionotophoresis through microdevices, and direct deposition of fluid, solids, or other dosing forms into the skin. The invention encompasses conventional injection needles, catheters or microneedles of all known types, employed singularly or in multiple needle arrays.

In a preferred embodiment, when the administration to the junctional layer involves needles, the needle front tip is preferably at 2.5 mm from the skin surface, and the bevel heel at 2 mm from the skin surface. Preferably, in order to accurately and selectively target the junctional layer, the needle is inserted at a perpendicular angle to the skin surface. The bevel size from the tip of the needle to the heel of the bevel must be in the range of 0.55 to 0.60 mm for delivering a substance to the junctional layer.

The junctional administration methods of the present invention result in an improved pharmacokinetics (PK) and pharmacodynamics (PD) obtained from a substance. By “improved pharmacokinetics,” it is meant that an enhancement of pharmacokinetic profile is achieved as measured, for example, by standard pharmacokinetic parameters such as time to maximal plasma concentration (Tₘₐₓ), the magnitude of maximal plasma concentration (Cₘₐₓ) or the time to elicit a minimally detectable blood or plasma concentration (T_{lag}). By “enhanced absorption profile,” it is meant that absorption is improved or greater as measured by such pharmacokinetic parameters. The measurement of pharmacokinetic parameters and determination of minimally effective concentrations are routinely performed in the art. Values obtained are deemed to be enhanced by comparison with a standard route of administration such as, for example, subcutaneous administration or intramuscular administration. In such comparisons, it is preferable, although not necessarily essential, that administration into the junctional layer and administration into the reference site such as subcutaneous administration involve the same dose levels, i.e., the same amount and concentration of drug as well as the same carrier vehicle and the same rate of administration in terms of amount and volume per unit time. Thus, for example, administration of a given pharmaceutical substance into the junctional layer at a concentration such as 100 μg/ml and rate of 100 μl per minute over a period of 5 minutes would, preferably, be compared to administration of the same pharmaceutical substance into the subcutaneous space at the same concentration of 100 μg/ml and rate of 100 μl per minute over a period of 5 minutes.

The above-mentioned PK and PD benefits are best realized by accurate direct targeting of the junctional layer. This is accomplished, for example, by using microneedle systems of less than about 250 micron outer diameter, and less than 5 mm, preferably less than 3 mm, exposed length. The preferred delivery device for use in the methods of the invention is a 30G, 2 mm needle length assembled to a syringe as drug reservoir. Such systems can be constructed using known methods of various materials including steel, silicon, ceramic, and other metals, plastic, polymers, sugars, biological and or biodegradable materials, and/or combinations thereof.

It has been found that certain features of the junctional administration methods provide clinically useful PK/PD and dose accuracy. For example, it has been found that placement of the needle outlet within the skin significantly affects PK/PD parameters.

Another benefit of the invention is that the risk of back pressure is reduced relative to ID delivery when delivering a substance to the junctional layer. Although not intending to be bound by a particular mechanism of action, this may be, in part, due to the lack of elastin fibers in the junctional layer.

Typically, the injection time for delivering 100 to 120 μL by ID route as a bolus is in the range of 8 to 15 seconds due, in part, to the intrinsic elasticity of the dermal compartment. The maximal injection volume to the dermal compartment ranges from 50 μL to 250 μL depending on the body site. In contrast to ID delivery, the injection time for junctional delivery is quicker, i.e., below 10 sec, and the maximal injection volume is higher than 250 μL, regardless of the site of injection.

The administration methods useful for carrying out the invention include both bolus and infusion delivery of a substance of the present invention to humans or animals subjects. A bolus dose is a single dose delivered in a single volume unit over a relatively brief period of time, typically less than about 10 minutes. Infusion administration comprises administering a fluid at a selected rate that may be constant or variable, over a relatively more extended time period, typically greater than about 10 minutes. To deliver a substance the junctional-access means is placed adjacent to the skin of a subject providing directly targeted access within the junctional layer and the substance or substances are delivered or administered into the junctional layer where they can act locally or be absorbed by the bloodstream and be distributed systematically. The junctional-access means may be connected to a reservoir containing the substance or substances to be delivered.

Delivery from the reservoir into the junctional layer may occur either passively, without application of the external pressure or other driving means to the substance or substances to be delivered, and/or actively, with the application of pressure or other driving means. Examples of preferred pressure generating means include pumps, syringes, elastomer membranes, gas pressure, piezoelectric, electromotive, electromagnetic pumping, or Belleville springs or washers or combinations thereof. If desired, the rate of delivery of the substance may be variably controlled by the pressure-generating means. As a result, the substance enters the junctional layer and is absorbed in an amount and at a rate sufficient to produce a clinically efficacious result.

As used herein, the term “clinically efficacious result” is meant a clinically useful biological response including both diagnostically and therapeutically useful responses, resulting from administration of a substance of the present invention. For example, diagnostic testing or prevention, management or treatment of a disease or condition is a clinically efficacious result.

5.4 Devices for Junctional Delivery

The present invention encompasses any device for accurately and selectively targeting the junctional layer of a subject's skin. The nature of the device used is not critical as long as it penetrates the skin of the subject to the targeted depth within the junctional layer without passing through it. Preferably the device penetrates the skin at a depth of at least about 2 mm, up to a depth of no more than about 3 mm, most preferably, no more than about 2.5 mm. The invention compasses drug delivery devices and needle assemblies disclosed in U.S. Pat. No. 6,494,865 and U.S. patent application Ser. Nos. 10/357,502 and 10/337,413 (filed on Feb. 4, 2003 and Jan. 7, 2003, respectively), all of which are incorporated herein by reference in their entireties. Once one skilled in the art is armed with the knowledge of the instant application relating to the boundaries of the junctional layer, the devices disclosed in the above-identified patent and applications may be modified using routine experimentation to be adapted for junctional delivery.

The invention encompasses conventional injection needles, catheters or microneedles of all known types, employed singularly or in multiple needle arrays. Alternatively, the invention encompasses needle-less devices including ballistic injection devices. As used herein, the terms “needle” and “needles” are intended to encompass all such needle-like structures. Examples include, but are not limited to, conventional injection needles, cannulas, catheters or microneedles of all known types. The term “microneedles,” as used herein, are intended to encompass structure 30 gauge and smaller, typically about 31-50 gauge when such structures are cylindrical in nature. Non-cylindrical structures encompass by the term microneedles would therefore be of comparable diameter and include pyramidal, rectangular, octagonal, wedged, and other geometrical shapes.

The present invention encompasses delivering a substance into the junctional layer using a device that comprises at least one needle, preferably a microneedle. Preferably, the needle has a length sufficient to penetrate the junctional layer and an outlet at a depth within the junctional layer so that the substance is delivered and distributed in the junctional layer. In some embodiments, the length of the needle is about 2 mm to about 5 mm, preferably about 2 mm to about 3 mm. In other embodiments, the outlet of the needle is placed at a depth of about 2 mm to about 3 mm, preferably about 2 mm to about 2.5 mm, when the needle is inserted. Preferably however, the device has structural means for controlling skin penetration to the desired depth within the junctional layer. This is most typically accomplished by inserting the microneedle at a perpendicular angle from the skin surface until the needle penetration in the skin is limited by either the needle hub or alternatively by a component preventing deeper penetration of the needle. The component can be part of the microneedle cannula or an assembled component. The length of microneedles for junctional delivery are easily varied during the fabrication process and are routinely produced in less than 3 mm length. The microneedle can be a disposable needle, which is assembled to a drug container such as for instance a syringe, or can be a pre-attached needle in the tip of a syringe. Microneedles used in the methods of the invention are also very sharp and of a very small gauge such as 30 or 34 G, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of substance delivered in a given period of time. Microneedles may be incorporated into a variety of devices such as holders and housings that may also serve to limit the depth of penetration. The junctional delivery devices of the invention may also incorporate reservoirs to contain the substance prior to delivery or pumps or other means for delivering the drug or other substance under pressure. Alternatively, the junctional delivery devices may be linked externally to such additional components.

In one embodiment, the device of the present invention for junctional delivery comprises the following components: a needle having a length sufficient to penetrate the junctional layer and an outlet at a depth within the junctional layer so that the substance is delivered and distributed in the junctional layer; a structure for loading, storing and dispensing the substance; and a structure for controlling skin penetration to the desired depth within the junctional layer. In one embodiment, the structure for loading, storing and dispensing the substance is a syringe. In another embodiment, the structure is an automated injector, such as, but not limited to, a pen, gun or an auto-injector. The structure for controlling the skin penetration may function to allow a perpendicular needle insertion into the skin of the subject.

In one embodiment, the device of the present invention has structural means for loading, storing and/or dispensing the formulations containing a substance of the present invention, i.e., a reservoir. Delivery from the reservoir into the junctional layer may occur either passively, without application of the external pressure or other driving means to the substance or substances to be delivered, and/or actively, with the application of pressure or other driving means. Examples of preferred pressure generating means include pumps, syringes, elastomer membranes, gas pressure, piezoelectric, electromotive, electromagnetic pumping, or Belleville springs or washers or combinations thereof. Specifically, a syringe or an automated injector such as, but not limited to, a pen, gun or an auto-injector may be advantageously used in accordance with the present invention.

If desired, the rate of delivery of the substance may be variably controlled by the pressure-generating means. As a result, the substance enters the junctional layer and is absorbed in an amount and at a rate sufficient to produce a clinically efficacious result. In some embodiments, the rate and volume of the delivery may be automatically controlled by employing an algorithm that has logic components. Examples of such algorithms include, but are not limited to, physiological models, rules based models or moving average method, therapy pharmacokinetic models, monitoring signal processing algorithms, predictive control models and combinations thereof.

In one embodiment, the device has structural means for controlling skin penetration to the desired depth within the junctional layer. This is most typically accomplished by means of a widened area or hub associated with the shaft of the junctional-access means that may take the form of a backing structure or platform to which the needles are attached. The length of needles as junctional-access means are easily varied during the fabrication process and are routinely produced in less than 5 mm, preferably less than 3 mm, length. Needles are also a very sharp and of a very small gauge, to further reduce pain and other sensation during the injection or infusion. They may be used in the invention as individual single needles or multiple needles may be assembled or fabricated in linear arrays or two-dimensional arrays as to increase the rate of delivery or the amount of substance delivered in a given period of time. Needles may be incorporated into a variety of devices such as holders and housings that may also serve to limit the depth of penetration. The device housing the junctional-access means may be linked externally to additional components, such as a reservoir and a control means for administration volume and rate.

The methods of the invention also include ballistic fluid injection devices, powder-jet delivery devices, piezoelectric, electromotive, electromagnetic assisted delivery devices, gas-assisted delivery devices, which directly penetrate the skin to provide access for delivery or directly deliver substances to the targeted location within the junctional layer.

5.5 Determination of Therapeutic Efficacy

The therapeutic efficacy of formulations containing a substance of the present invention may be determined using any standard method known to one skilled in the art or described herein. The assay for determining the therapeutic efficacy of the formulations of the invention may be in vivo or in vitro based assays, including animal based assays. Preferably, the therapeutic efficacy of the formulations of the invention is done in a clinical setting.

In some embodiments, the pharmacokinetic and pharmacodynamic parameters of the delivery of a substance of the invention is determined, preferably quantitatively using standard methods known to one skilled in the art. In preferred embodiments, the pharmacodynamic and pharmacokinetic properties of a substance of the invention, delivered using the methods of the invention, are compared to those of the substance delivered by other conventional modes of administration, e.g., subcutaneous or intramuscular delivery, to establish the therapeutic efficacy of the substance administered in accordance with the methods of the invention. Pharmacokinetic parameters that may be measured in accordance with the methods of the invention include but are not limited to Tₘₐₓ, Cₘₐₓ, T_{lag}, AUC, etc. Other pharmacokinetic parameters that may be measured in the methods of the invention include for example, half-life (t_{1/2}), elimination rate constant and partial AUC values. Standard statistical tests which are known to one skilled in the art may be used for the statistical analysis of the pharmacokinetic and pharmacodynamic parameters obtained.

In a specific embodiment, the invention encompasses determining the therapeutic efficacy of a substance administered in accordance with the methods of the invention by comparing the pharmacokinetic profile to that of, for example, subcutaneous or intramuscular delivery. An exemplary assay for determining the therapeutic efficacy of a substance may comprise the following: administering a substance of the present invention with a 30G, 1.5 mm needle; or a 34G, 2 mm needle, with a 34G, 3 mm needle, or subcutaneous or intramuscular injection to humans. Preferably ⅝ inch 25G needle is used for intramuscular injection. Injection volume of 0.2 mL is used for junctional and subcutaneous delivery, and 0.5 mL is used for intramuscular delivery. In the case of vaccines, booster injections are given at day 0, 7 and 21. The blood samples are collected and preferably centrifuged at 3000 rpm for a period of at least fifteen minutes at a temperature between 2 to 8° C., within one hour of sample collection. The serum from the collection tube is transferred for analysis of serum levels.

The present invention can be further illustrated by the following, non-limiting examples.

### 6. EXAMPLES

6.1 Junctional Delivery of Enoxaparin®

Enoxaparin® in 2,000 and 4,000 aXa IU preparation, was injected to a subject's junctional layer. The junctional injection of Enoxaparin® yielded Tₘₐₓ of 1.5 to 2.3 hours, which is faster than that obtained from subcutaneous injection, indicating a faster onset of Enoxaparin® when junctionally administered. Cₘₐₓ value (0.2 to 0.6 aXa IU/mL) and overall AUC (2 to 4.5 aXa IU/mL/hour) were substantially the same for both junctional and subcutaneous injections.

6.2 Junctional Delivery of Fondaparinux®

Fondaparinux® was injected to a subject's junctional layer. The junctional injection of Fondaparinux® yielded Tₘₐₓ of 1.5 to 2.3 hours, indicating a faster onset of Enoxaparin® when junctionally administered compared to subcutaneous injection. Cₘₐₓ value (0.3 to 0.45 mg/mL) and overall AUC were substantially the same for both junctional and subcutaneous injections.

6.3 Open Randomized Pilot Study in Healthy Human Volunteers of Rabies Virus Vaccination

The primary objective of this study was to investigate the impact of delivery depth on the immune response against rabies vaccine by assessing the antibody response against the rabies antigen. The rabies vaccine was delivered to sero-negative human volunteers and the depth of delivery was varied using needles of different lengths. The trial design was an open study, randomized with parallels groups.

Subjects: 10 subjects per group including males and females, aged 18 to 40 years were enrolled. The inclusion and non-inclusion criteria is set forth in Tables 1 and 2, infra.

TABLE 1
Inclusion Criteria for Subjects

Healthy male and female Caucasian subjects aged 18-40 years,
Subjects must have major organ functions within acceptable medical limits
as determined by the clinical history and the physical examination,
Subjects must have a normal blood pressure and heart rate measured
under standardized conditions at the screening visit after at least 5 minutes
of rest in a supine position: SBP within 90 to 140 mmHg, DBP within 40
to 85 mmHg and HR within 40 to 85 bpm,
Subject must have a normal 12-lead electrocardiogram at screening
recorded after at least 5 minutes of rest: PR within 120 to 200 ms,
QRS ≦ 120 ms and QTc ≦ 440 ms. Incomplete right bundle branch block
will be accepted,
Subjects must have laboratory results within the normal ranges or
considered not being of clinical relevance by the Investigator,
Subjects must be sero-negative against rabies vaccine antigens,
Subjects must agree not to take any medicine which can impact the
rabies antibody rise in the blood after immunization.
Female subjects must be using an adequate birth control method (hormonal
treatment or intrauterine device) to prevent pregnancy for at least 3 months
before and following the immunization in the study and must have a
negative urine pregnancy test at screening,

TABLE 2
Non-Inclusion Criteria for Subjects

Subjects with a known history of allergy or hypersensitivity to any
component of the rabies vaccine (albumin, neomycin),
Subjects with a known immunodeficiency, systemic cancer, or use
of immunosuppressive medication including cancer chemotherapy
and systemic steroids,
Subjects with an active dermatologic disease,
Subjects with type I diabetes or other major illness,
Subjects with a mild upper and lower respiratory illness, ENT local
infection, gastro-intestinal illness or other febrile episode that is expected
and documented to resolve will be temporarily excluded,
Subjects with symptomatic or asymptomatic orthostatic hypotension at
screening defined by a decrease of SBP or DBP by more than 20 mmHg,
between supine and standing position,
Subjects who have a positive urine drug screening (cannabinoids,
benzodiazepines),
Subjects who are pregnant or breast feeding,
Subjects with a level of antibody upper 0.5 UI/ml against rabies antigens
in the blood at the time of the screening period,
Subjects with an history of hepatitis B or C and/or positive results from the
hepatitis serology which indicate an acute or chronic hepatitis B or C,
Subjects with a positive HIV serology,
Subjects who have given more than 400 ml of blood within the last
three months,
Subjects who are participating in another clinical study within 30 days
of enrollment into this study.
Subjects with excessive body hair on the upper outer arm,

Investigational Drug: Purified Vero Cells Rabies Vaccine (PVRV) was used in this study. This vaccine is manufactured from Vero cells by Aventis Pasteur (France) and has been shown to be safe and effective for prevention and treatment of rabies in humans. Numerous studies have reported on the safety and efficacy of the intramuscular delivery of PVRV. In addition, intradermal delivery of rabies vaccine has also been thoroughly investigated in humans and subjected to expert panel review. Intradermal delivery route of rabies vaccine is recognized to be as effective and safe as the intramuscular route for pre and post-exposure immunization. The main benefit of the intradermal route is the antigen dose sparing, e.g., 1/10 the intramuscular dose, which represents a substantial cost reduction for developing countries where rabies disease remain a public health issue.

Due to the high antigenic effect of PVRV rabies antigens, all young healthy volunteers are vaccine responders without a risk of any serious adverse events as documented by post-immunization follow-up in clinical trials. Thus, PVRV rabies vaccine is considered as a safe and reliable immuno-pharmacological model in clinical research. In addition to the safety profile of the vaccine there are other advantages for choosing PVRV rabies vaccine as a clinical investigation model in volunteers, including its strong antigenic effect in adults with about 100% of responder to the vaccine. Furthermore, because rabies immunization is not a mandatory vaccination in France, most of the French population is sero-negative to PVRP antigens.

Investigational Devices: Four different devices were used in this study:

As a positive control for intramuscular injection (IM) a needle of 16 mm length was used. In this case, the dose of vaccine delivery per injection was 0.5 mL. For intradermal injection (ID) a 30G, 1.5 mm needle was used where the dose of vaccine delivery per injection was 0.2 mL. For junctional injection (JI) a 34G needle of 2 mm and 3 mm needle length was used and the dose delivered per injection was the same as that used for ID delivery.

Study Design: 10 adults per treatment group, healthy volunteers, females and males were enrolled by treatment groups; Group I: intradermal using 1.5 mm needle; Group II: junctional using 2 mm needle; Group III: junctional using 3 mm needle; Group IV: IM using 16 mm needle.

The purpose of the investigation was to investigate the differences between the immune response as measured by circulating antibody titer during the induction phase of immune response. Therefore, antibody titer from D0 to D14 resulting from a single dose of rabies vaccine were compared. The anti-rabies immunization regimen consisted of three subsequent injection at D0, D14 and D21. The measurements of antibody titers were done at D0, D7, D14, D21 and D49 post-immunization.

Results:

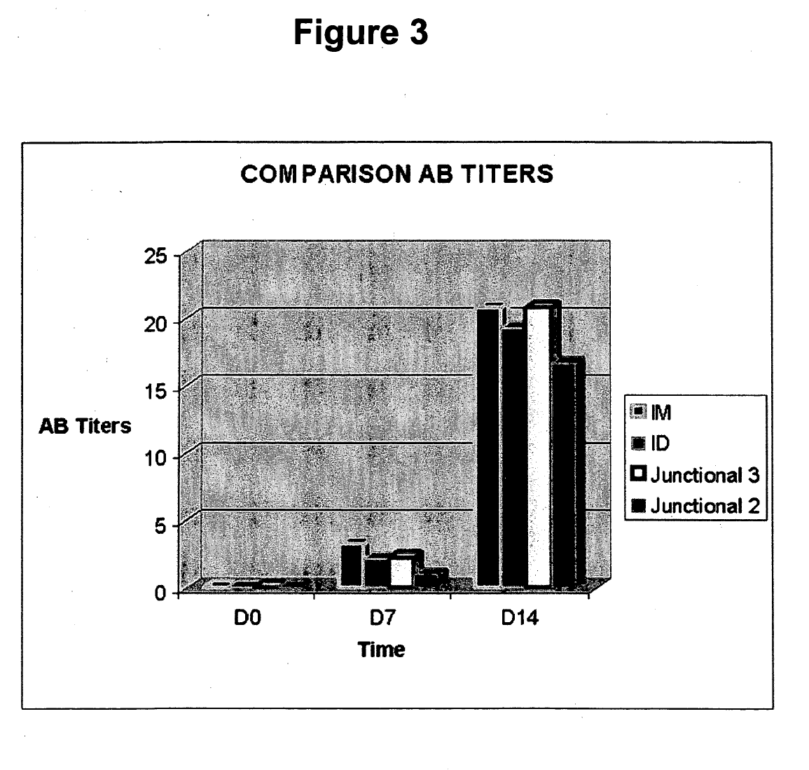

FIG. 3 and Table 3 show the geometric mean titers (GMT) of antibody after a single injection of rabies vaccine. Junctional injection using 2 mm needle length yielded a lower GMT than the other delivery routes. This lower GMT value at D7 from junctional injection is probably due to a delayed immune response when the antigen is delivered to the junctional layer at a depth of 2 mm.

Considering that rabies vaccine is a very powerful antigen, the lower GMT at D7 and D14 with junctional delivery at a depth of 2 mm suggests that the junctional layer is less reactive than the dermis and the muscle in inducing an immune response against the rabies viral antigen. Delivery at a depth of 3 mm, however, is equivalent to intradermal delivery in terms of the magnitude of the immune response generated.

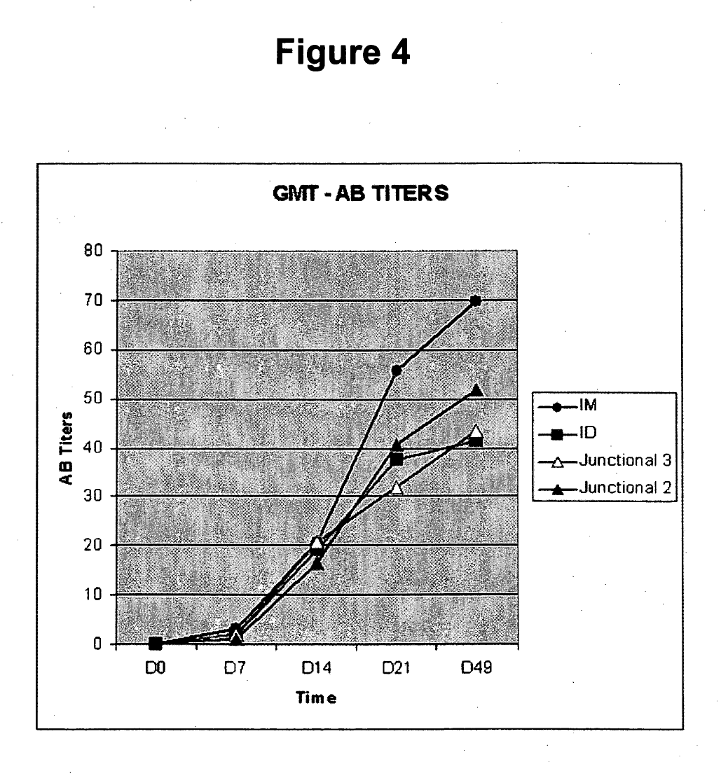

After two and three subsequent injections the immune response is not significantly impacted by the delivery route (FIG. 4).

TABLE 3
GEOMETRIC MEAN TITERS (GMT) OF ANTIBODY AFTER A
SINGLE INJECTION OF RABIES VACCINE

| | | | | | |
|---|---|---|---|---|---|
| IM | 0 | 3.27 | 20.8 | 55.8 | 69.74 |
| ID | 0 | 2.12 | 19.27 | 37.45 | 41.44 |
| Junctional 3 | 0 | 2.22 | 20.79 | 31.99 | 43.19 |
| Junctional 2 | 0 | 1.03 | 16.73 | 40.53 | 51.82 |

While the invention has been described with respect to the particular embodiments, it will be apparent to those skilled in the art that various changes and modifications may be made without departing from the spirit and scope of the invention as recited by the appended claims.

1. A method for administration of a substance into a subject's skin, comprising delivering the substance into a junctional layer of the subject's skin.

2. The method of claim 1, wherein the subject is a human or an animal.

3. The method of claim 2, wherein the subject is a human.

4. The method of claim 1, wherein the administration comprising using a device comprising at least one needle.

5. The method of claim 4, wherein the needle has a length sufficient to penetrate the junctional layer and an outlet at a depth within the junctional layer so that the substance is delivered and distributed in the junctional layer.

6. The method of claim 5, wherein the needle is about 2 mm to 5 mm long.

7. The method of claim 6, wherein the needle is about 2 mm to 3 mm long.

8. The method of claim 5, wherein the outlet is at a depth of about 2 mm to 2.5 mm when the needle is inserted.

9. The method of claim 4, wherein the substance is a liquid delivered by pressure directly on the liquid.

10. The method of claim 4, wherein the substance is injected as a bolus.

11. The method of claim 9 or 10, wherein the substance is delivered in a volume of 0.5 ml or greater.

12. The method of claim 11, wherein the substance is delivered in a volume of 1 ml or greater.

13. The method of claim 4, wherein a single needle is used.

14. The method of claim 4, wherein multiple needles are used.

15. The method of claim 4, wherein the needle is a microneedle, a catheter needle or an injection needle.

16. The method of claim 1, wherein inadvertent immune response caused by the substance is minimized.

17. The method of claim 16, wherein the substance is not a vaccine.

18. The method of claim 1, wherein the substance is a low molecular weight heparin, a pentasaccharide, interferon alpha, interferon beta, a erythropoeitine, a monoclonal antibody, a polypeptidic hormone or an interleukin.

19. The method of claim 18, wherein the substance is a low molecular weight heparin or a pentasaccharide.

20. The method of claim 19, wherein the low molecular weight heparin is Enoxaparin.

21. The method of claim 19, wherein the pentasaccharide is Fondaparinux.

22. A device for delivering a substance into a junctional layer of a subject comprising:
(a) a needle having a length sufficient to penetrate the junctional layer and an outlet at a depth within the junctional layer so that the substance is delivered and distributed in the junctional layer;
(b) a structure for loading, storing and dispensing the substance; and
(c) a structure for controlling skin penetration to the desired depth within the junctional layer.

23. The device of claim 22, wherein the needle is about 2 mm to 5 mm long.

24. The device of claim 23, wherein the needle is about 2 mm to 3 mm long.

25. The device of claim 22, wherein the outlet is at a depth of about 2 mm to 2.5 mm when the needle is inserted.

26. The device of claim 22, which has a single needle.

27. The device of claim 22, which has multiple needles.

28. The device of claim 22, wherein the structure is for loading, storing and dispensing the substance is a syringe.

29. The device of claim 22, wherein the structure for loading, storing and dispensing the substance is an automated injector.

30. The device of claim 22, which further comprises a means for controlling the rate and volume of delivery.
